(19) United States
(12) Patent Application Publication
(10) Pub. No.: US 2002/0076695 A1
(43) Pub. Date: Jun. 20, 2002

(54) METHODS FOR TREATING PROSTATE CANCER

(76) Inventor: JEFFREY S. ROSS, NEW LEBANON, NY (US)

Correspondence Address: Kathy Smith Dias, Heslin & Rothenberg, 5 Columbia Circle, Albany, NY, 12203, US

(21) Appl. No.: 09/152,934

(22) Filed: Sep. 14, 1998

Related U.S. Application Data
(63) Continuation-in-part of application No. 09/088,417, filed on Jun. 1, 1998.
Application No. 09/088,417 is a continuation-in-part of application No. 08/832,745, filed on Apr. 4, 1997.

(30) Foreign Application Priority Data
Apr. 3, 1998 (US) PCTUS9806621

(51) Int. Cl. C12Q 1/68

(52) U.S. Cl. 435/6

(57) ABSTRACT

A method for treating a patient afflicted with prostate cancer by determining whether the number of copies of HER-2/neu gene in prostate cells from the patient exceeds four by in-situ hybridization and treating such patient having prostate cells with five or more copies of the HER-2/neu gene with an anti-HER2/neu antibody alone or in combination with an anti-androgen.

This application is a continuation-in-part of U.S. Ser. No. 09/088,417, filed Jun. 1, 1998, which is a continuation-in-part of U.S. Ser. No. 08/832,745, filed Apr. 4, 1997, the contents of which are hereby incorporated by reference in their entirety into the subject application.

### FIELD OF THE INVENTION

The present invention relates to treatment of neoplastic disease and more particularly to treatment for prostate cancer.

### BACKGROUND OF THE INVENTION

Prostate cancer is the most common cancer in American men and the second leading cause of cancer death. The disease is responsible for nearly 3% of all deaths in men over the age of 55 years and it is likely that more than 300,000 new cases of prostate cancer will be diagnosed in American men this year.

Traditional therapies for prostate cancer include observation, radiation therapy, and radical prostatectomy, with radiation being the definitive choice because of the high disease-specific survival rates. More recently, treatment with anti-androgens has been used and new cancer-specific therapeutic products are currently being developed expanding the spectrum of potential treatments.

Prostate cancer arises in a tissue whose development and function is strongly dependent upon hormones. Not surprisingly, therefore, these tumors are frequently stimulated by hormones, in particular androgens which are steroid hormones of high potency such as testosterone. Removal or inhibition of androgens plays a major role in therapy of this malignancy for some patients and this characteristic has been used in the design of endocrine manipulative therapy for prostate cancer. Hormonal manipulation is design primarily to decrease serum androgens or their effects and has been reported to effect improvement in symptoms of up to 80% of patients with prostate carcinoma. The primary aim of such treatments is to deprive the prostatic tumor of trophic androgens. When effective, hormone manipulations cause tumor regression in two ways: (i) by removing hormones that directly stimulate tumor growth, and (ii) by blocking production or release of other trophic factors that are direct stimuli for tumor growth. Two potent anti-androgens, cyproterone acetate and flutamide, have been used to treat patients with prostatic cancer. These agents have multiple effects, but their principal action is to inhibit binding of testosterone or dihydrotestosterone to the androgen receptor.

Unfortunately, following hormonal treatment resistance to the drug develops, particularly in metastatic disease or when diagnosed earlier in life, sometimes the tumors eventually regrow. In such a situation, the tumors are unresponsive to hormonal manipulation. Furthermore, many tumors that arise in hormone-responsive tissues do not respond to hormonal manipulations at all.

In current urologic practice, a subset of men often diagnosed early, before the age of 60, will either present with metastatic disease or relapse after primary surgery or radiation treatment. These men initially respond to anti-androgen therapy, but ultimately become insensitive to the treatment. It has been known for decades that most prostate cancer metastasize will initially regress if they are deprived of androgen stimulation. originally, anti-androgen therapy consisted either of bilateral orchiectomy (castration) or exogenous administration of diethyl stilbestrol (estrogens). In the last ten years, new anti-androgen drugs were developed that either block androgen receptor(s) or are lutenizing hormone releasing factor antagonists.

A small percentage of anti-androgen treated men are refractory to therapy at the onset. Most responded initially and if they were going to become hormone treatment resistant, developed symptomatic metastasize and/or rising serum PSA (prostate specific antigen) levels after months or years of hormone-based therapy. It is assumed that either new genetic mutations occur in the “dormant” metastatic sites that confers the ability for the tumor to grow again in the absence of androgen stimulation or that clones of hormone-independent tumor that were “masked” by the initially faster growing hormone dependent cells. The hormone-independent tumor cells are then permitted to grow without competition as these hormone dependent cells are suppressed by the anti-androgen therapy.

Accordingly, there is a clear need for adjuvant or alternative therapeutic approaches to prostate tumors that grow independent of androgens and are therefore not fully responsive to hormonal manipulations.

Prostate cancer has variable clinical outcome and recent studies indicating the potential benefits of withholding therapy in older men with limited disease and the potential to predict inoperable cancer in men with aggressive tumors has prompted the search for new prognostic markers that would be applied to the initial guided prostate needle biopsy and prove successful in selecting therapy and predicting disease outcome. Since severe patient distress can be caused by more aggressive therapy regimens, it is desirable to determine when such therapies are warranted. For example, patients with a high likelihood of relapse can be treated aggressively with powerful systemic chemotherapy and/or radiation therapy. Where there is a lesser likelihood of relapse, less aggressive therapies can be chosen. It is also desirable to identify those patients who might be candidates for newly developed target-specific therapies such as those described herein. There is thus a clear need for new assays to predict which tumors would likely respond to particular treatment regimes such as the aforesaid adjuvant or alternative therapeutic approaches thereby allowing an attending physician to select the most appropriate course of therapy.

It has been hypothesized that the use of anti-androgens early in disease treatment, i.e. in a neoadjuvant approach prior to prostatectomy, hasten the development of the androgen-independent clones. Thus, a marker that could predict the “risk” that such a treatment with existing or future anti-androgen resistant tumors would be of significant clinical value.

### SUMMARY OF THE INVENTION

In one aspect, the invention relates to a method for treating prostate cancer by using compositions that block expression of the HER-2/neu oncogene or function of the gene product. The HER-2/neu protein is a cell membrane tyrosine kinase that is a member of the epidermal growth factor receptor family. These compositions, which may include for example antibodies, vaccines, and gene therapy approaches, would be preferably employed in those tumors wherein the HER-2/neu gene is amplified. Similar therapeutic approaches have been successfully employed in battling breast cancer. See Drebin et al, Cell 42: 695-706 (1985), Drebin et al, Oncogene 2: 273-277 (1988), Drebin et al, Oncogene 2: 387-394 (1988) and Fendley et al, Cancer Research 50:1550-1558 (1990). However, it was heretofore unknown and unexpected that such agents could be used against prostate tumors. See Baselga et al, Journal of Clinical Oncology 14: 737-744 (1996), Cobleigh et al, Proc. ASCO 17:97a (1998) and Slamon et al, Proc. ASCO 17:98a (1998).

The reported data relating to prostate cancer and HER-2/neu has been extremely variable with a number of “negative” studies concerning prognostic significance. However, these studies have involved detecting HER-2/neu protein not the gene. See Vaiskorpi et al, Modern Pathology 5: 643-648 (1992), Ibriham et al, surgical oncology 1: 151-155 (1992), Kuhn et al, Journal of Urology 150: 1427-1433 (1993), Sadasivan et al, Journal of Urology 150: 125-131 (1993) and Ware et al, Human Pathology 22: 254-258 (1991).

In a related aspect, the invention relates to the combination of anti-androgen therapy and an inhibitor of HER2/neu to treat prostate cancer. Such anti-androgen therapy could be, for example, Casodex™ (bicalutamide), Eulexin™ (flutamide), Lupron™ (leuprolide acetate), Zoladex™ (goserelin), estrogens, destruction or removal of androgen producing cells from the body, such as orchiectomy and combinations of these.

In a related aspect, the invention relates to a method for testing for HER-2/neu gene amplification and treating prostate cancer using anti-androgen therapy in patients which do not have the HER-2/neu gene amplified.

In another aspect, the invention relates to a method for selecting treatment for prostate cancer based on the determination that the number of copies of HER-2/neu gene in prostate cells from the patient. When the number of copies is abnormally high aggressive therapy is indicated and treatment with anti-androgen therapy is contraindicated unless combined with an inhibitor of HER-2/neu.

### DESCRIPTION OF THE PREFERRED EMBODIMENTS

The term “prostate cancer” includes adenocarcinoma of the prostate, particularly when infiltrating the prostate stroma, prostatic epithelial neoplasia, and metastasis thereof regardless of their location.

An “anti-androgen” is an effective chemical or surgical treatment which reduces the amount of androgens in the blood, reduces the effect of androgens on prostate cells (especially prostate cancer) or acts on the cells to have an effect contrary or annulling the effect of an androgen. Examples of reducing blood concentrations include surgical removal (or destruction chemically or immunologically) of androgen producing cells such as orchiectomy or by addition of a composition. Examples of compositions include: estrogens such as diethyl stilbestrol, Lupron™ (leuprolide acetate) is a nonapeptide analog of naturally occurring gonadotropin releasing hormone (gn-RH or LHRH), thereby suppressing testicular steroidogenesis by acting as an LHRH agonist and Zoladex™ (goserelin), a synthetic decapeptide analog of LHRH which leads to suppression of pituitary gonadotropins after sustained administration. It is believed to act as a potent inhibitor of pituitary gonadotropins upon sustained administration. Other techniques include removal with a hormone adsorbent or degrading enzyme or other agent. Examples of agents which reduce the effect of the androgen include Casodex™ (bicalutamide), which inhibits the action of androgens by binding to cytosol androgen receptors in the target tissue and Eulexin™ (flutamide) which inhibit androgen uptake and/or by inhibiting nuclear binding of androgen to a receptor in target tissues or both. Contrary effecting compositions include estrogens.

An “anti-HER-2/neu composition” includes compositions which act on the HER-2/neu DNA, the HER-2/neu mRNA (spliced or not), the HER-2/neu protein or inhibit or counter the activity of the HER-2/neu protein. Examples of a composition which acts on the DNA and RNA include anti-sense oligonucleotides or triple strand forming oligonucleotides which code for a DNA or RNA complementary to and capable of binding HER-2/neu mRNA or HER-2/neu DNA, thereby preventing its transcription, splicing or translation into protein. Ribozymes may also be used which catalytically alter the HER-2/neu gene or mRNA. Examples of compositions which act on the protein include antibodies, fragments thereof, or other protein binding agents to the HER-2/neu protein; peptides which exhibit sufficient homology to the tyrosine kinase growth factor ligand to bind to and inactivate the HER-2/neu protein; antagonizing analogs to the HER-2/neu receptor; small molecule signal transduction inhibitors (Sugen, Inc.), a vaccine or other immunological preparations containing a chemical moiety resembling the HER-2/neu protein and capable of eliciting an immune response (antibodies or cellular immunity) against the HER-2/neu protein and enzymes which modify the protein by cleavage, altered glycosylation or altered three dimensional configuration. An example of a composition which counters the action of HER-2/neu is a drug with an antigrowth activity. Preferred compositions are a recombinant humanized monoclonal antibody such as Herceptin™ (Genentech, South San Francisco) and MDX-210 (Medarex), a bispecific antibody combination with the capability of directly linking the body's immune cells to the target cancer cells. A more complete description of Herceptin™ is found is found in Hudziak et al, U.S. Pat. No. 5,725,856. Other antibodies to HER-2/neu and their uses are described above. Alternative treatments may include any therapeutic products designed to attack breast cancer cells expressing elevated levels or gene copy numbers of HER-2/neu. At the present time, no effective chemotherapy has been established. However, if chemotherapy were effective, it could also be administered in combination with, subsequent to or prior to the above treatment.

To detect HER-2/neu overexpression, one may assay for an excess amount of the HER-2/neu protein by immunoassay or other diagnostic protein assay such as gel electrophoresis. However, these techniques give results which are highly variable and do not measure the prognosis for prostate cancer as noted above. It may be possible to detect overexpression of HER-2/neu by measuring HER-2/neu mRNA. However, RNA is easily degraded and difficult to quantitatively measure.

In the present invention, the number of copies of the HER-2/neu gene are detected. This may be done by a number of techniques, the easiest of which is by in-situ hybridization. Normal cells contain 2 copies of each gene. After DNA replication and just before cell division, a cell may have 4 copies of a gene. The detection of five or more copies of the HER-2/neu gene clearly indicates the presence of amplified HER-2/neu genes.

Fluorescence in-situ hybridization (FISH) and other gene detection methods may be used in accordance with the present invention to detect amplification of HER-2/neu genes in prostate tissue and provide a reliable technique for identifying suitable candidates for anti-HER-2/neu treatment. Identification of an amplified HER-2/neu status very early in the diagnostic process followed by treatment with anti-HER-2/neu treatment, such as a anti-HER-2/neu antibody-based compositions or a gene therapy which utilizes an anti-sense gene to inhibit HER-2/neu, can prevent the progression of the disease to more advanced stages. Additionally, patients who have an androgen-independent form of the disease derive a clinical benefit from the administration of an anti-HER/2-neu treatment in combination with anti-androgen.

In accordance with the present invention increased copy number of the HER-2/neu gene in prostate tissues is detected using FISH techniques. The structure of the HER-2/neu gene is well known. See, e.g., King et al., Science, 229:974-978 (1985) and Coussens et al., Science, 230:1132-1139 (1986). Detectable DNA probes capable of hybridizing to the known HER-2/neu gene sequence are constructed and labeled using conventional techniques. See, for example, PCT Application Pub. No. WO94/09022, the entire contents of which are incorporated herein by reference. Examples of labeling systems include those which incorporate digoxygenin, biotin, avidin, streptavidin and antibodies. Labeled DNA probes are then allowed to hybridize to available HER-2/neu genes and are detected using conventional fluorescence detecting techniques such as fluorescence microscopy, spectrophotometers, fluorescent plate readers and flow sorters. For signal detection, fluorescent molecules can be linked directly to the DNA probe or can be linked to a binding partner for the probe. Useful fluorescent molecules include, but are not limited to fluorescein, amino coumarin acetic acid, tetramethylrhodamine isothiocyanate, Texas Red, Cy3.0, Cy5.0, and green fluorescent protein. Other non-fluorescent labels may be used such as chemiluminescent, radioactive, enzyme, ligand, spin labels, quenchers etc., and the choice is well known and within the skill of the art. The selection among known labels in the DNA hybridization and other binding assays (e.g. immunoassay) Signal detection and amplification techniques known to those skilled in the art can be utilized in accordance with the present invention. Thus, signal detection and amplification techniques such as those involving streptavidin/biotin, avidin/biotin, hapten conjugates such as digoxigenin/anti-digoxigenin, dinitrophenyl and other known antibody based detection and amplification techniques are utilized herein.

Amplification of HER-2/neu correlates to a decreased chance of long term survival as well as a shortened time to relapse of the disease. Determination of the HER-2/neu copy number in the prostate cells from an initial needle biopsy in accordance with the present invention can be used to identify patients with a biologically aggressive form of prostate cancer. The expected number of signals in a normal cell and in an unamplified tumor cell varies from 2 to 4 depending on the phase of the cell cycle. A cell with five or more signals is considered amplified. Individuals with cells in which amplification of the HER-2/neu gene is observed may require different or more aggressive treatment.

Conversely, patients having prostate cancer with a low copy number of HER-2/neu can be treated with milder conventional therapy, such an anti-androgen treatment alone, to lessen or avoid adverse side effects while containing the cancer or placed under observation thereby avoiding radiation or drug exposure entirely until such time as some therapeutic intervention is absolutely indicated.

In addition to traditional treatments like surgical intervention, and higher doses of radiation, alternative methods of treatment for prostate and other cancers are being developed. Alternative treatments may include therapeutic products designed to attack specific cancer cells. Specifically, compositions directed against cancers which exhibit overexpression of the HER-2/neu protein would be desirable. Such compositions include antibodies, or fragments thereof, to the HER-2/neu protein and peptides which exhibit sufficient homology to the tyrosine kinase growth factor ligand. These compositions may be linked to a marker moiety, which is readily recognized as foreign by the patient, and cytotoxic moieties (e.g. ricin chain) or structures (liposomes, etc. containing a drug).

Prostate cancer is known to be androgen sensitive and generally responds to treatment that counteracts the effect of androgen and/or removes the source of androgen in many cases. Castrate levels of testosterone can be achieved with surgical orchiectomy or by administration of a an anti-androgen. An anti-androgen is any substance which inhibits the synthesis or action of androgen. There are several anti-androgenic compounds currently in clinical use. Diethylstilbestrol (DES), an estrogenic compound providing therapeutic responses similar to natural estrogens, Casodex™ (bicalutamide), for example, inhibits the action of androgens by binding to cytosol androgen receptors in the target tissue. Eulexin™ (flutamide 750 mg/day orally in three dosages) exerts its anti-androgenic action by inhibiting androgen uptake and/or by inhibiting nuclear binding of androgen in target tissues or both. Lupron™ (leuprolide acetate 1 mg/day by injection) is a nonapeptide analog of naturally occurring gonadotropin releasing hormone (gn-RH or LHRH), and, therefore, acts as an LHRH agonist suppressing ovarian and testicular steroidogenesis. Similarly, Zoladex™ (goserelin), a synthetic decapeptide analog of LHRH acts as a potent inhibitor of pituitary gonadotropins. Sustained administration of Zoladex™ leads to suppression of pituitary gonadotropins; as a result, serum levels of testosterone fall into the range normally seen with surgical castration 2-4 weeks after initiation of therapy.

Unfortunately, some prostate carcinomas eventually become refractory in the presence of anti-androgen, that is, their growth becomes androgen-independent. The HER-2/neu gene amplification rate for men with progressive hormone refractory disease was about 67%, nearly twice the rate of amplification seen in the non-hormone refractory group.

Thus, a composition which acts on cells having amplified HER-2/neu compensates for a weakness in anti-androgen therapy, namely resistance related to amplified HER-2/neu. Combination therapy acts on the prostate cancer cells from two opposing mechanisms of action. Cells resistant to one therapy are believed to do so by mutating or being selected to be susceptible to the other therapy. Thus, their use in combination is desirable, particularly in view of the prognosis for prostate cancer.

While advanced prostate cancers are typically the most demanding in treatment effectiveness, the treatments of the present invention may also be used for early stage prostate cancers. Since earlier treatment is generally more successful than when the patient is terminal, early treatment, even at the point of initial diagnosis is within the present invention.

Anti-HER-2/neu and anti-androgen compositions used are pharmaceuticals (biologicals, e.g. vaccines, are considered pharmaceuticals) and typically are mixed with a vehicle or carrier and which are pharmaceutically acceptable. The nature of the pharmaceutically acceptable carrier or vehicle, its selection and formulation based on active ingredient and route of administration is well known to those skilled in the art.

The two compositions may be mixed in the same container, unitary dosage or they be in separate containers. In either situation, a kit may be formed containing one or more of the compositions along with instructions for usage treating prostate cancer. The kit may be in a number of different configurations such as one or more containers in a single box or other manner linking the two compositions in close proximity to each other. Also, the linkage may be indirect by way of the instructions contained in packages of one or both drugs.

The following examples are included for purposes of illustrating certain aspects of the invention and should not be construed as limiting.

### EXAMPLE 1

### Fish Assay for HER-2/NEU

Samples of prostate carcinoma taken during biopsy or after surgical removal of suspected prostate cancer were selected without regard to the clinical history of the patients or the outcome of any therapy.

The assay was performed by the Inform® HER-2/neu Gene Detection System (FDA approved version). Briefly, unstained four micron formalin-fixed paraffin-embedded tissue sections were applied to silanized slides and processed according to the Oncor chromosome in-situ hybridization system (Oncor, Inc., Gaithersburg, Md.). Briefly, tissue deparaffinization in xylene was followed by transfer through two changes of 100% ethanol and the slides were allowed to air dry. The slides were then immersed for 30 minutes in 30% Oncor pretreatment solution (30% sodium bisulfite in 2× SSC (0.45 molar NaCl and 0.045 molar sodium citrate)) at 45° C. and 45 minutes in Oncor protein digesting solution (0.25 mg/ml proteinase K in 2× SSC) at 45° C. After a brief wash in 2× sodium chloride/sodium citrate (SSC) slides were dehydrated in 100% ethanol and allowed to air dry. Oncor unique sequence digoxigenin-labeled HER-2/neu DNA probe consisting of 4 contiguous overlapping cosmid probes which create a 90 kb unbroken DNA strand (available from Oncor, Inc. Catalog Nos. P5111-BIO, P5111-DIG, P5111-B.5, P5111-DG.5, S8000-KIT or S8000-KIT-E) was prewarmed for five minutes at 37° C. prior to application. The amount of probe hybridization mixture was approximated according to the target area and the size of the coverslip to be placed over the tissue during hybridization (10 Al probe mixture per 22× 22 mm coverslip area). Denaturation was accomplished at 69° C. for five minutes and the slides were then incubated overnight at 37° C. in a pre-warmed humidified chamber. Following overnight hybridization slides were again immersed in 2× SSC and pre-warmed to 72° C. for a five minute stringency wash in 40 ml 2× SSC at pH 7.0 prior to detection. Fluorescein-labeled anti-digoxigenin (commercially available from Boerhinger Mannheim) in a solution containing 5% nonfat dry bovine milk, 0.08% sodium azide, 0.05% NP-40, 0.1 molar NaH₂PO₄ and 0.1 molar K₂H₂PO₄ was applied and a plastic coverslip placed gently for a 20 minute incubation at 37° C. in a pre-warmed humidified chamber in the dark. After careful removal of the coverslip and rinsing of excess detection compounds in 1× phosphate-buffered detergent (PBD) for three rinses at two minutes each, slides were counterstained with 18 Al of propidium iodide/antifade (1:4) and covered with a glass coverslip. Slides were evaluated for HER-2/neu gene copy number using a Zeiss Axioskop 50 fluorescence microscope.

The probe displays a single fluorescent signal at the location of each copy of the HER-2/neu gene. The expected number of signals in a normal cell and in an unamplified tumor cell varies from 2-4 depending on the phase of the cell cycle. A cell with five or more signals was considered amplified. A minimum of 100 tumor cells in each prostate carcinoma specimen was evaluated for the number of nuclear HER-2/neu signals. Amplified tumors were defined as having a minimum of 20 cells with five signals or greater per cell. The number of signals was not averaged between cells.

### EXAMPLE II

### HER-2/NEU Gene Copy Number and Treatment with Anti-Androgens

To investigate the relationship between HER-2/neu gene amplification and androgen-independent prostate cancer, prostate tissue specimens from patients by needle biopsy, or other tissue removal, who had undergone hormone therapy were examined. In the group that received hormone therapy, 50 mg Casodex™ (bicalutamide) was administered daily to patients in combination with an LHRH agonist Eulexin™ (flutamide 750 mg/day orally) to maintain androgen at castrate levels. Patients in a refractory group who experienced progression of the prostate cancer in the absence of hormone treatment were evaluated as well. Serum PSA levels and bone pain index were used as indicators of progression of the disease.

From the cohort, 220 men with prostate cancer were selected. All of the men had surgery and the removed tumor tissue was assayed for number of copies of the HER-2/neu gene. HER-2/neu gene amplification was determined by FISH, as in Example 1. All men were placed on anti-androgen therapy of 50 mg Casodex™ (bicalutamide) daily.

From the cohort, 20 of the men with prostate cancer had a rising PSA level on this anti-androgen therapy. These patients are considered non-responders. HER-2/neu was amplified in the primary tumor of 14 (70%) of these men.

200 men with prostate cancer have a stable PSA level on this anti-androgen therapy. These patients are considered responders. This group included both “cured” and relapse patients whose cancer remains sensitive to anti-androgen therapy. HER-2/neu was amplified in the primary tumor of 35% of these men.

### EXAMPLE III

### HER-2/neu Copy Number and Patient Therapy Response to Recombinant Humanized Monoclonal Antibody to HER-2/neu

To evaluate the effectiveness of recombinant humanized monoclonal antibody to HER-2/neu treatment in older prostate cancer patients with advanced stage disease, disease progression despite treatment with anti-androgens, and no history of other malignancy, except non-melanoma skin cancer, the following study is performed.

To participate in the study, patients must demonstrate progression of adenocarcinoma of the prostate, while on the primary anti-androgen hormone therapy. Progressive disease is defined by evidence of new osseous lesions as detected by bone scan. Alternatively, evidence of a greater than 25% increase in bidimensionally measurable soft tissue disease constitutes progression. An increasing serum PSA despite maintenance of castrate levels of testosterone also is indicative of progression of the disease. Additionally, to rule out patients experiencing an improvement due to anti-androgen withdrawal, patients in the study were required to show progression of the disease and be off of the anti-androgen therapy for at least four weeks prior to enrollment.

Biopsy or surgically removed tumor cells are assayed for HER-2/neu amplification using the technique of Example 1.

Patients receive 4 mg/kg Herceptin™ (Genentech, Inc., South San Francisco, Calif.) administered IV over 90 minutes, as a loading dose on Day 0. Subsequently, 2 mg/kg is administered IV over 30 minutes weekly for up to 24 weeks, or until disease progression or unacceptable side effects necessitated removal of the patient from the study.

The results of this study indicate whether treatment with Herceptin™ bestows a clinical benefit by slowing or halting the progression of the prostate cancer. Additionally, administration of Herceptin™ as a single agent confers a survival advantage in prostate cancer patients whose prostate cells from the initial needle biopsy demonstrated HER-2/neu amplification as compared to patients without HER-2/neu amplification.

Early intervention by identifying the prostate cancer patient at the time of the initial needle biopsy as being HER-2/neu amplified and beginning Herceptin™ treatment is expected to enhance the patients chances of survival by slowing or stopping progression of the disease while avoiding treatment in individuals who will not significantly benefit from Herceptin™ treatment.

### EXAMPLE IV

### Treatment of Prostate Cancer With Anti-HER-2/NEU and Anti-Androgen

The techniques of Example III are repeated on another group of 15 prostate cancer patients filling the same criteria. In TM addition to Herceptin™ treatment, the patients are simultaneously treated with anti-androgen therapy of 50 mg Casodex™ (bicalutamide), and Eulexin™ (flutamide 750 mg/day orally) daily.

The results of this study indicate whether combined treatment with an anti-HER-2/neu composition and an anti-androgen treatment bestows a clinical benefit by slowing or halting the progression of the prostate cancer over Herceptin™ as a single agent.

The effectiveness of this treatment indicates that earlier treatment of prostate cancer at the time of the initial needle biopsy, indicating HER-2/neu amplified, with an anti-HER-2/neu treatment and an anti-androgen treatment is expected to enhances the patients chances of survival by slowing or stopping progression of the disease.

### EXAMPLE V

### Treatment of Prostate Cancer With a HER-2/NEU Vaccine With Optional Anti-Androgen Treatment

The techniques of Example II are repeated on another group of 25 prostate cancer patients filling the same criteria. However instead of receiving Herceptin™ treatment, the patients are vaccinated with a HER-2/neu vaccine previously proposed for treating breast cancer. 0.2 ml of this vaccine contains about 1 mg HER-2/neu protein previously produced by recombinant NIH 3T3 cells containing an expression vector with the cloned HER-2/neu gene. 10 of the patients simultaneously receive the anti-androgen therapy of 50 mg Casodex™ (bicalutamide) and Eulexin™ (flutamide 750 mg/day orally) daily.

Response is quantified by measuring serum PSA levels weekly for at least 12 weeks. While not actually used in this Example for technical reasons, the formation of anti-HER-2/neu antibody and/or cellular immune response in the patient's serum may be measured, correlated to all other medical data and used as a predictor of prognosis or determination of further treatment also.

The results of this study indicate whether the vaccine alone or combined with an anti-androgen treatment bestows a clinical benefit by slowing or halting the progression of the prostate cancer.

The effectiveness of this treatment indicates that earlier treatment of prostate cancer at the time of the initial needle biopsy, indicating HER-2/neu amplified, with a vaccine to HER-2/neu alone or with an anti-androgen treatment is expected to enhance the patients chances of survival by slowing or stopping progression of the disease.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

What is claimed is:

1. A method for treating prostate cancer comprising: administering to a patient in need of such treatment a therapeutically effective amount of an anti-HER-2/neu composition.

2. The method of claim 1 wherein said anti-HER-2/neu composition comprises an antibody or fragment thereof.

3. The method of claim 2 wherein said anti-HER-2/neu antibody is a recombinant humanized monoclonal antibody.

4. The method of claim 1 wherein said anti-HER-2/neu composition comprises a peptide or protein.

5. The method of claim 1 wherein the patient has a HER-2/neu gene copy number exceeding 4 copies per cell.

6. The method of claim 1 wherein said anti-HER-2/neu composition comprises an oligonucleotide.

7. The method of claim 1 further comprising administering an effective anti-androgen therapy to the patient.

8. The method of claim 1 wherein the prostate cancer is an androgen-independent prostate cancer.

9. The method of claim 7 wherein the anti-androgen exerts its anti-androgenic action by inhibiting androgen receptors or by inhibiting production of hormones.

10. The method of claim 9 wherein the anti-androgen is selected from the group consisting of flutamide, leuprolide acetate, bicalutamide or goserelin.

11. The method of claim 4 wherein the anti-androgen protein or peptide is a vaccine.

12. A composition comprising:
(a) an anti-HER-2/neu composition, and
(b) an anti-androgen.

13. The composition according to claim 12 wherein the anti-HER-2/neu composition is a recombinant humanized monoclonal antibody.

14. A therapeutic kit comprising:
(a) a container containing at least one dose of an anti-HER-2/neu composition and
(b) a container containing at least one dose of an anti-androgen,
wherein said dose is an effective amount for treating prostate cancer.

15. The kit according to claim 12 wherein the anti-HER-2/neu composition is a recombinant humanized monoclonal antibody.

16. A method for determining therapy for a patient with prostate cancer comprising determining whether cells of the prostate cancer contain an abnormally elevated number of copies of HER-2/neu gene wherein an abnormally elevated number indicates therapy with an anti-HER-2/neu composition and a normal number indicates no therapy with an anti-HER-2/neu composition.

17. The method of claim 16 wherein the abnormally elevated number is at least 5.

18. The method of claim 16 wherein a normal number indicates therapy with an anti-androgen.

19. The method of claim 16 wherein an abnormally elevated number indicates therapy with both an anti-HER-2/neu composition and an anti-androgen.
